(19) United States
(12) Patent Application Publication
(10) Pub. No.: US 2024/0091388 A1
(43) Pub. Date: Mar. 21, 2024

(54) Process for Controlled Manufacturing of Mono-Disperse Microbubbles

(71) Applicant: Solstice Pharmaceuticals B.V., Enschede (NL)

(72) Inventors: Miguel de Vargas Serrano, Enschede (NL); Giuliana Manzi, Enschede (NL)

(21) Appl. No.: 18/259,201

(22) PCT Filed: Dec. 23, 2021
(86) PCT No.: PCT/NL2021/050785

§ 371 Date: Jun. 23, 2023

(30) Foreign Application Priority Data
Dec. 27, 2020 (NL) 2027237

(51) Int. Cl. A61K 49/22

(52) U.S. Cl. CPC A61K 49/223

(57) ABSTRACT

The present invention relates to a process for preparing a hydrated phospholipids solvent mixture, by: —dissolving a first phospholipid at a temperature above the phase transition temperature of the phospholipids in an organic solvent to form a dissolved phospholipid solvent mixture; —dissolving a second phospholipid at a temperature above the phase transition temperature of the phospholipids in the dissolved phospholipid solvent mixture to form a dissolved phospholipids solvent mixture; —adding an aqueous phosphate buffer to the dissolved phospholipids solvent mixture to form a buffered phospholipids solvent mixture; and —stirring the buffered phospholipids solvent mixture to form a hydrated phospholipids solvent mixture.

### FIELD OF THE INVENTION

The present invention relates to a process for preparing a phospholipid composition and the product thereof. It is furthermore related to the use of the phospholipid composition in the controlled manufacturing of mono-disperse microbubbles. Such phospholipid compositions are widely used to create microbubbles to create ultrasound contrast agent microbubbles.

### BACKGROUND OF THE INVENTION

In ultrasound imaging, the size and quantity of the bubbles is of utmost importance. They are for example being used to increase the contrast in ultrasound imaging. These bubbles have a high degree of echogenicity, which is the ability of an object to reflect the ultrasound waves. The bubbles are administered intravenously or into a body cavity (intracavitary administration, e.g. in urine for visualization of the reflux of urine) allowing for instance the blood flow through organs to be visualized with high contrast. The size of the bubbles determines their resonance frequency and thereby their acoustic properties, whereas the quantity of bubbles should be sufficient to achieve suitable contrast while not causing health risks for the patient. Bubbles are created using phospholipid compositions. Bubbles have a gas core and a phospholipid shell. Phospholipid compositions are known in the art.

During ultrasound examination, the operator of the ultrasound imaging apparatus determines the desired frequency of the ultrasound waves with which the examination should be performed. This frequency is determined by the depth of the tissue or organ to be analysed. Typically, higher frequencies are used for superficial body structures and lower frequencies are used for deeper body structures. To achieve a suitable contrast, it is desired that the resonance frequency of the microbubbles corresponds to the desired frequency. Moreover, the variance in resonance frequencies among the microbubbles should be sufficiently low.

Microbubbles generally comprise a shell that is filled by a gas core. The combination of the gas core and the shell determine the resonance frequency of the microbubble. When the microbubble is subjected to an ultrasound wave of a suitable frequency, for example, equaling or at least approaching the resonance frequency of the microbubble, the bubble will resonate at the resonance frequency of the microbubble. It is also possible to insonify the microbubbles at twice their resonance frequency, for example to stimulate sub-harmonic emissions. The resonance can be picked up by the ultrasound imaging apparatus. Moreover, when a mono-disperse microbubble is subjected to an ultrasound wave of an a priori known frequency its acoustic frequency response is reproducible and accurately predictable. In this manner, a high contrast can be achieved between microbubble-rich and microbubble-poor regions.

U.S. Pat. No. 9,801,959 describes a composition for stabilizing a fluorocarbon emulsion. The composition includes phosphatidylcholine, phosphatidylethanolamine-PEG, and a cone shaped lipid. The composition comprises no phosphatidic acid DPPA. They describe that DPPA catalyzes or accelerates the hydrolysis of the lipids in the formulation. They furthermore describe that a cone shaped lipid, in particular DPPE, provide better bubble count and better microbubble stability than without such a third cone-shaped lipid.

U.S. Pat. No. 9,545,457 describes the preparation of a lipid blend and a phospholipid suspension containing the lipid blend, which is useful as an ultrasound contrast agent.

A disadvantage of the above lipid blends is that they are less suitable for microfluidic systems. A further disadvantage is that it is difficult to scale up, one of the reasons being the use of toxic organic solvents to prepare a mixture of lipids. Another disadvantage is the presence of toxic organic solvent traces in a final product.

Accordingly, there is a demand for alternative preparation processes for phospholipid compositions to produce phospholipid compositions that are more stable and simpler to produce. There is furthermore a demand for phospholipid compositions with a higher concentration of phospholipids, which can be suitably used in microsystems. There is also a demand for simplification of the process to produce phospholipid compositions without the use of toxic organic solvents.

### SUMMARY OF THE INVENTION

It is an object of the present invention to provide a process for the preparation of a phospholipid composition that has a better stability in general. It is furthermore an object of the present invention to provide a process for the preparation of a phospholipid composition that is suitable for use in microfluidic systems. It is a further object of the present invention to prepare phospholipid compositions without the use of toxic organic solvents. It is a further object of the present invention to develop a practical manufacturing process of a lipid formulation that is totally biocompatible. It is yet another object of the present invention to develop a practical manufacturing process that can be easily scaled up. It is also an object of the invention to develop a process that leads to the formation of uniform filterable phospholipid solutions. It is yet a further objective of the present invention to prepare a phospholipid composition that prevents coalescence of microbubbles during their (microfluidic) manufacturing to obtain a uniform size distribution.

Accordingly, the present invention relates to a process for preparing a phospholipid composition, by:
dissolving a first phospholipid at a temperature above the phase transition temperature of the phospholipid in an organic solvent to form a dissolved phospholipid solvent mixture;
dissolving a second phospholipid at a temperature above the phase transition temperature of the phospholipid in the dissolved phospholipid solvent mixture to form a dissolved phospholipids solvent mixture;
adding an aqueous phosphate buffer to the dissolved phospholipids solvent mixture to form a buffered phospholipids solvent mixture; and
stirring the buffered phospholipids solvent mixture to form a hydrated phospholipids solvent mixture.

The present invention also relates to the phospholipid compositions prepared by the process of this invention.

The present invention furthermore relates to the use of phospholipid composition in a system for controlled manufacturing of microbubbles.

### DETAILED DESCRIPTION OF THE INVENTION

Unless otherwise defined, all technical and scientific terms used herein have the same meaning as commonly understood by one of ordinary skill in the art which this invention belongs. The terminology used in the description of the invention herein is for the purpose of describing particular embodiments only and is not intended to be limiting of the invention.

The term “microbubbles” as used herein, includes bubbles that essentially display the same resonance frequencies and are also referred to as mono-disperse microbubbles.

The term “mono-disperse” as used herein, includes to characterize a collection of microbubbles, and may be construed to mean that the poly-dispersity index (PDI) of the collection, mathematically defined as PDI=s/n, wherein n denotes the average bubble radius and s the standard deviation of the bubble radii, is smaller than 5×10⁻². That is, a collection of bubbles having a PDI <10% may be considered to be mono-disperse. Within the context of the present invention, microbubbles are bubbles having a diameter of below and including 10 micrometer and preferably in the range of from 2 up to and including 5 micrometer. Bubbles with larger diameters than 10 micrometer may not safely flow through the smallest capillaries of a patient's blood vessel system and provoke oedema. On the other hand, smaller bubbles may possess poor ultrasound reflectivity.

The term “dispersed phase fluid” as used herein, includes one or more gases from the group consisting of SF₆, N₂, CO₂, O₂, H₂, He, Ar, ambient air, and perfluorocarbon gases, such as CF₄, C₂F₆, C₂F₈, C₃F₆, C₄F₈, C₄F₆, C₄F₈, C₄F₁₀, C₅F₁₀, C₅F₁₂ and mixtures thereof.

Microbubbles generally comprise a shell that is filled by a gas core. The combination of the gas core and the shell determine the resonance frequency of the microbubble. When the microbubble is subjected to an ultrasound wave of a suitable frequency, equaling or at least approaching the resonance frequency of the microbubble, the bubble will resonate at the resonance frequency of the microbubble. This resonance can be picked up by the ultrasound imaging apparatus. In this manner, a high contrast can be achieved between microbubble-rich and microbubble-poor regions.

A microbubble generation unit is known from WO-A-2016118010. The contents of this patent application are hereby incorporated by reference, for all purposes.

The term “phase transition temperature of the phospholipids” as used herein, includes the temperature required to induce a change in the lipid physical state from the ordered gel phase, where the hydrocarbon chains are fully extended and closely packed, to the disordered liquid crystalline phase, where the hydrocarbon chains are randomly oriented and fluid.

The term “phospholipids” or “lipids” as used herein, includes a class of lipids whose molecule has a hydrophilic “head” containing a phosphate group, and two hydrophobic “tails” derived from fatty acids, joined by an alcohol residue. The phosphate group can be modified with simple organic molecules such as choline, ethanolamine or serine. Phospholipids are a key component of all cell membranes. They can form lipid bilayers because of their amphiphilic characteristic. In eukaryotes, cell membranes also contain another class of lipid, sterol, interspersed among the phospholipids. The combination provides fluidity in two dimensions combined with mechanical strength against rupture.

The term “non-toxic solvent” as used herein, includes a class of solvents that are non-hazardous to the health of living creatures, such as humans and animals. Examples are (but not limited to) propylene glycol, ethylene glycol, water, various phosphate buffers etcetera.

The present invention is a novel and green process for preparing a phospholipid composition. It is a practical manufacturing process of a lipid formulation that is totally biocompatible, can be easily scaled up and most importantly that leads to the formation of uniform filterable phospholipid solutions. The solution is ready to use for microbubble formation using microfluidic flow focusing technology. It is preferred that coalescence is absent during bubble formation.

Dissolving of the lipids is executed by weighing out the required amounts preferably at room temperature. If required, lipids are defrosted first. Then, lipids are dissolved one by one in a flask, preferably with a preheated organic solvent, more preferably with a preheated non-toxic organic solvent, at a temperature above the phase transition temperature of the phospholipids. A next lipid is only added to the mixture once the previous lipid is preferably completely dissolved in the (non-toxic) organic solvent. With completely dissolved in the (non-toxic) organic solvent is being meant that at least 80 wt % of the lipid is dissolved, preferably at least 90 wt % is dissolved, more preferably at least 95 wt % is dissolved, even more preferably at least 99 wt % is dissolved. With the temperature being above the phase transition temperature of the phospholipids is being meant that the temperature is above the phase transition temperature of the phospholipid with the highest phase transition temperature. It is possible to dissolve a first phospholipid at a temperature above the phase transition temperature of the first phospholipid in the organic solvent to form a dissolved phospholipid solvent mixture, followed by dissolving a second phospholipid at a temperature above the phase transition temperature of the second phospholipid in the dissolved phospholipid solvent mixture to form a dissolved phospholipids solvent mixture. However, it is preferred to use preheated organic solvent, even more preferred to use preheated non-toxic organic solvent, at a temperature above the phase transition temperature of the phospholipid with the highest phase transition temperature. Preferably, the preheated organic solvent is at a temperature above 65° C., more preferably above 70° C.

It is a further option to dissolve the lipids separately in separate flasks with the solvent, at a temperature above the phase transition temperature of the phospholipids and then add the dissolved lipid solutions together to form one dissolved phospholipids solvent mixture. This is however not the preferred route.

In for example U.S. Pat. No. 9,801,959 the preparation of the mixture of lipids is different from our first steps, as a mixture of lipids is dissolved in propylene glycol. Traditionally, liposomal solutions of a mixture of lipids are prepared following the Bangham method, mostly known as thin-film hydration method (Bangham et al., J. Mol. Biol. 1965, 13: 238). Briefly, this procedure consists of the solubilization of phospholipid solid mixtures in organic solvents (i.e. chloroform and methanol). Organic solvents are subsequently removed by evaporation under reduced pressure, thereafter the obtained thin film is added to propylene glycol and hydrated with an aqueous buffer. A disadvantage of this procedure is that toxic solvents might be present in the end product. Post-treatments for removing traces of organic solvents are required, as well as additional clinical test for proving that the product is not toxic.

Solvent systems used in lipid suspension are classified as either aqueous or non-aqueous vehicles. Choice of a typical solvent system depends on solubility and long-term stability of the final formulation. The organic solvent used in the present invention to dissolve the lipids in, is preferably selected from the group of propylene glycol, ethylene glycol, polyethylene glycol 3000 and/or glycerol, more preferably the organic solvent is propylene glycol. These organic solvents are classified as non-aqueous water miscible agents, and are used as co-solvents. They are furthermore non-toxic. The use of propylene glycol, also referred to as PG, 1,2-propanediol or propane-1,2-diol, an organic compound (diol or double alcohol) with formula C₃H₈O₂ is most preferred as it is a clear, colorless, viscous liquid, hygroscopic and miscible with water. PG is most preferably used in this instance for acting as a co-solvent in order to improve the solubility of phospholipid compounds. Clinically, the use of PG as an excipient in marketed products is generally well tolerated. It is preferably used in the range of from 5 up to 60% V/V.

In the next step, an aqueous phosphate buffer is added to the dissolved phospholipids solvent mixture to form a buffered phospholipids solvent mixture. The aqueous phosphate buffer is preferably phosphate buffered saline (PBS), phosphate buffered saline with glycerine, water, saline, saline/glycerine and/or a saline/glycerine/non-aqueous solution, more preferably phosphate buffered saline (PBS). It is most preferred to use a combination of propylene glycol (PG) as the non-aqueous and non-toxic solvent, combined with phosphate buffered saline (PBS), is selected in order to adjust and stabilize the pH of the mixture close to the physiologic one.

The ratio of solvent to buffer (in the most preferred case PBS/PG) is preferably in the range of from 80/20% V/V, more preferably in the range of from 90/10% V/V up to 98/2% V/V. It is most preferred to have a final liquid composition of 95/5% V/V PBS/PG±1.5 V/V PBS/PG.

A preferred phospholipid according to the invention is chosen from the group of DPPC, DSPC, DSPG, DMPC, DBPC, DPPE, DPPE-mPEG5000, DMPE-PEG-2000 and DSPE-PEG2000. More preferably, the phospholipids are a combination of at least one out of the group of DPPC, DSPC, DSPG, DMPC, DBPC, DPPE and at least one out of the group of DPPE-mPEG5000, DMPE-PEG-2000 and DSPE-PEG2000, even more preferably one out of the group of DPPC, DSPC, DPPE and one out of the group of DPPE-mPEG5000, and DSPE, most preferably DPPC and DPPE-mPEG5000. DPPC is the most preferred lipid as it was observed in for example single-microbubble dissolution studies, that microbubbles coated with DPPC remained smooth. Furthermore, DPPC offered no measurable resistance to surface shear and oxygen gas permeation. From the group of DPPE-mPEG5000, DMPE-PEG-2000 and DSPE-PEG2000, DPPE-mPEG5000 is preferred as this is an excellent lipopolymer emulsifier.

Advantageously, the ratio of the lipids when 2 lipids are present in the hydrated phospholipids solvent mixture is in the range of from 95:5 to 70:30, more preferably in the range of from 90:10 to 75:25, even more preferably in the range of from 85:15 to 80:20.

Advantageously, sequentially one or more phospholipids might be dissolved in the dissolved phospholipids solvent mixture at a temperature above the phase transition temperature of the phospholipids. Thus an end product comprising more than two lipids is preferably anticipated in this invention. As additional lipid, a bifunctional PEG'ylated lipid may be employed.

Bifunctional PEG'ylated lipids include but are not limited to DSPE-PEG(2000) Succinyl 1,2-distearoyl-sn-glycero-3-phosphoethanolamine-N-[succinyl(polyethylene glycol)-2000] (ammonium salt), DSPE-PEG (2000) PDP 1,2-distearoly-sn-glycero-3-phosphoethanolamine-N-[PDP(polyethylene glycol)-2000] (ammonium salt), DSPE-PEG (2000) Maleimide 1,2-distearoly-sn-glycero-3-phospho-ethanolamine-N-[maleimide (polyethylene glycol)-2000] (ammonium salt), DSPE-PEG(2000) Biotin 1,2-distearoyl-sn-glycero-3-phosphoethanolamine-N-[maleimide (polyethylene glycol)-2000] (ammonium salt), DSPE-PEG (2000) Cyanur 1,2-distearoly-sn-glycero-3-phosphoethanolamine-N-[cyanur (polyethylene glycol)-2000] (ammonium salt), DSPE-PEG(2000) Amine 1,2-distearoyl; -sn-glycero-3-phosphoethanolamine-N-[amino (polyethylene glycol)-2000] (ammonium salt), DPPE-PEG (5,000)-maleimide, 1,2-distearoyl-sn-glycero-3-phosphoethanolamine-N-[dibenzocyclooctyl (polyethylene glycol)-2000] (ammonium salt), 1,2-distearoyl-sn-glycero-3-phosphoethanolamine-N [azido(polyethylene glycol)-2000] (ammonium salt), 1,2-distearoyl-sn-glycero-3-phosphoethanolamine-N-[succinyl (polyethylene glycol)-2000] (ammonium salt), 1,2-distearoyl-sn-glycero-3-phosphoethanolamine-N-[carboxy (polyethylene glycol)-2000] (ammonium salt), 1,2-distearoyl-sn-glycero-3-phosphoethanolamine-N-[maleimide(polyethylene glycol)-2000] (ammonium salt), 1,2-distearoyl-sn-glycero-3-phosphoethanolamine-N-[PDP (polyethylene glycol)-2000] (ammonium salt), 1,2-distearoyl-sn-glycero-3-phosphoethanolamine-N-[amino (polyethylene glycol)-2000] (ammonium salt), 1,2-distearoyl-sn-glycero-3-phosphoethanolamine-N-[biotinyl (polyethylene glycol)-2000] (ammonium salt), 1,2-distearoyl-sn-glycero-3-phosphoethanolamine-N-[cyanur (polyethylene glycol) 2000] (ammonium salt), 1,2-distearoyl-sn-glycero-3-phosphoethanolamine-N-[folate (polyethylene glycol)-2000] (ammonium salt), 1,2-distearoyl-sn-glycero-3-phosphoethanolamine-N-[folate (polyethylene glycol)-5000] (ammonium salt), N-palmitoyl-sphingosine-1-{succinyl[methoxy (polyethylene glycol)2000]} and N-palmitoyl-sphingosine-1{succinyl[methoxy (polyethylene glycol)5000]}. The bifunctional lipids may be used for attaching antibodies, peptides, vitamins, glycopeptides and other targeting ligands to the microbubbles. The PEG chains MW may vary from about 1000 to about 5000 Daltons in the lipid.

According to the invention, it is preferred to perform all process steps at a temperature above the phase transition temperature of the phospholipids. The advantage of that is that the lipids are then homogenously mixed, as the lipids are all in the liquid crystalline phase during the whole process. The phase transition temperature is defined as the temperature required to induce a change in the lipid physical state from the ordered gel phase, where the hydrocarbon chains are fully extended and closely packed, to the disordered liquid crystalline phase, where the hydrocarbon chains are randomly oriented and fluid.

Advantageously, the buffered phospholipid propylene glycol mixture is stirred for at least 1 hour, more preferably for at least 2 hours, even more preferably for at least 4 hours, most preferably for at least 8 hours. In this step extensive hydration of the lipids occurs. The stirring step is an easy step when scaling up the procedure to larger batch sizes. Stirring can be done using a standard baffled mixer reactor.

Advantageously, the hydrated phospholipids solvent mixture is filtered over a sterilization filter to form a sterilized hydrated phospholipids solvent mixture. Contaminations are taken out of the phospholipids solvent mixture. More preferably, the sterilization filter has a pore size of 0.2 micrometer. To remove bacteria suspended in the solution, a 0.2 μm pore size is considered to be effective. With this filter also the bigger particles that may have influence in the microfluidic process of producing the microbubbles are being removed.

Advantageously, the hydrated phospholipids solvent mixture is filtered at least twice, more preferably it is filtered with a temperature of the mixture of above room temperature, more preferably above 50° C., and it is filtered with a temperature of the mixture of below room temperature, more preferably below 15° C. The second step is preferably done prior to storage of the hydrated phospholipids solvent mixture, resulting in a more stable mixture.

According to the invention, the concentration of the lipids in the hydrated phospholipids solvent mixture is in the range of from 5 up to 20 mg/ml, preferably in the range of from 10 up to 18 mg/ml. This higher concentration of the lipids in the hydrated phospholipids solvent mixture is advantageous for microfluidic manufacturing. These higher concentrations normally give problems with coalescence when “standard” phospholipic compositions are used. With the process of our invention these problems have been overcome. Coalescence of microbubbles resulting in polydisperse microbubble are no longer observed when the process of the present invention is applied. To maintain a monodisperse microbubble population coalescence should still be avoided.

The present invention is furthermore directed to a phospholipid composition obtainable by the process of the invention as described herein, wherein the total concentration of phospholipids is at least 12 mg/ml. Advantageously, the total concentration of the phospholipids is at least 15 mg/ml. This higher concentration of phospholipids is advantageous for microfluidic manufacturing. Higher concentration gives problems when dipalmitoylphosphatidic acid (DPPA) is present. Advantageously, the phospholipid composition according to the invention comprises no dipalmitoylphosphatidic acid (DPPA). When phospholipid compositions are prepared via the prior art processes, these higher concentrations give difficulties with coalescence. Coalescence of microbubbles results in polydisperse microbubble. To maintain a monodisperse microbubble population coalescence should be avoided.

Both the size and shell properties of the phospholipids are important for the behavior of the microbubbles. It is known in literature that microbubbles of the same size, but with different shell properties, behave differently. Advantageously, the phospholipid composition obtainable by the process of the invention as described herein comprises mono-disperse microbubbles with mean diameter between 1 and 10 μm, preferably between 2 and 5 μm. Alternatively defined, the phospholipid composition obtainable by the process of the invention as described herein comprises preferably microbubbles with a mono-dispersity PDI <=10%, which is in line with a geometric standard deviation (GSD)<=1.1. The process for preparing the phospholipid composition according to the present invention favours a more homogeneous liposome distribution resulting in more uniformity between microbubble shells.

During ultrasound examination, the operator of the ultrasound imaging apparatus determines the desired frequency of the ultrasound waves with which the examination should be performed. This frequency is determined by the depth of the tissue or organ and type of body structures to be analysed as well as the ultrasound procedure. To achieve a suitable contrast, it is desired that the resonance frequency of the microbubbles corresponds to the desired frequency. Moreover, the variance in resonance frequencies among the microbubbles should be sufficiently low. This is an example, however the insonation frequency may, for example, also be twice the resonance frequency of the microbubbles. It is desired that the variance in the acoustic behavior of the microbubbles is sufficiently low and predictable. To this end, controlled manufacturing of microbubbles is desired.

The present invention is also directed to a system for controlled manufacturing of microbubbles, comprising: a microbubble generation unit having a first inlet for receiving a dispersed phase fluid, a second inlet for receiving a continuous phase fluid, and a bubble formation channel in which microbubbles are generated using the received dispersed phase fluid and the received continuous phase fluid, wherein the continuous phase fluid is the phospholipid composition obtainable by the process of the invention as described herein. Microfluidic manufacturing gives uniform bubble size and uniformity of the shell properties. This improves uniform acoustic behaviour.

Preferably, the use of the microbubbles is also directed to therapeutic applications. For microbubble assisted drug delivery it is also important to have a predictable acoustic behaviour. This allows precise ultrasound triggering of microbubbles, hence improved control of drug or gene delivery. For non-invasive pressure estimation mono-disperse microbubbles with a well-defined acoustic behaviour improve the subharmonic signal leading to improved sensitivity of this measuring technique for a wide variety of clinical diseases.

Thus the present invention is also directed to the use of the phospholipid composition as described herein before in a system for controlled manufacturing of microbubbles. A short explanation: in microfluidics the microbubbles are produced using “flow focusing” a gas flow to flow through a narrow constriction. The inner gas is forced by the outer coflowing liquid flow to flow through a narrow constriction. In this constriction the gas flow forms a thin gaseous thread that breaks up into uniform microbubbles. The size of these microbubbles is governed by the gas-to-liquid flow rate ratio. Microbubbles are produced at typical production speeds between 100,000 and 1,000,000 microbubbles per second. Once the microbubbles are produced they decelerate and collide. This is related to the microfluidic method of producing the microbubbles. These collisions are violent and can cause coalescence (merging of two bubbles). This might be avoided by increasing the lipid concentration (to around 15 mg/mL, a ten-fold higher than “standard” lipid concentrations). Increasing the lipid concentration leads to problems for getting a homogeneous dispersion of liposomes. This has been solved by improving the preparation of the phospholipid composition and avoiding the use of DPPA. A high lipid concentration and the presence of DPPA leads to the formation of aggregates. Aggregates obstruct microfluidic production and results in poor filterability. Aggregates (or a poor homogeneity of the phospholipid composition) have a negative effect on the formation of the microbubble shell and cause the microbubble to be more likely to coalesce. Coalescence should be avoided to obtain monodisperse microbubbles.

The following non-limiting figures show the present invention further.

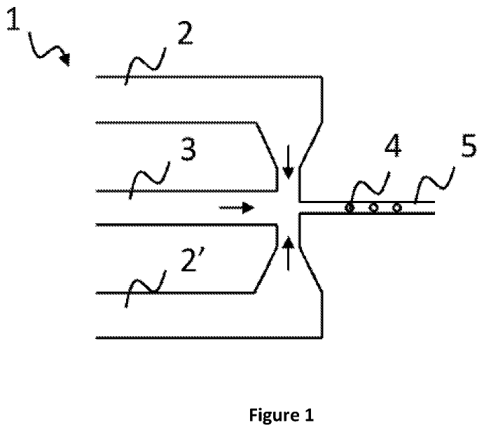

FIG. 1 illustrates a microbubble generation unit known from the art;

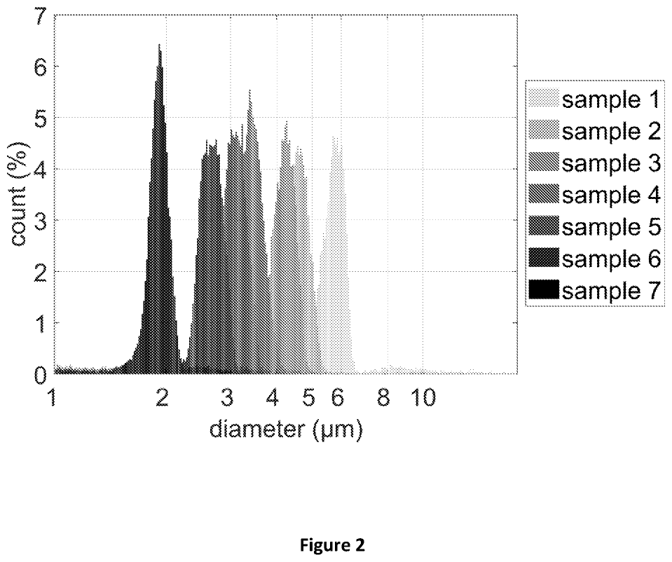

FIG. 2 illustrates the size distribution of different microbubble populations;

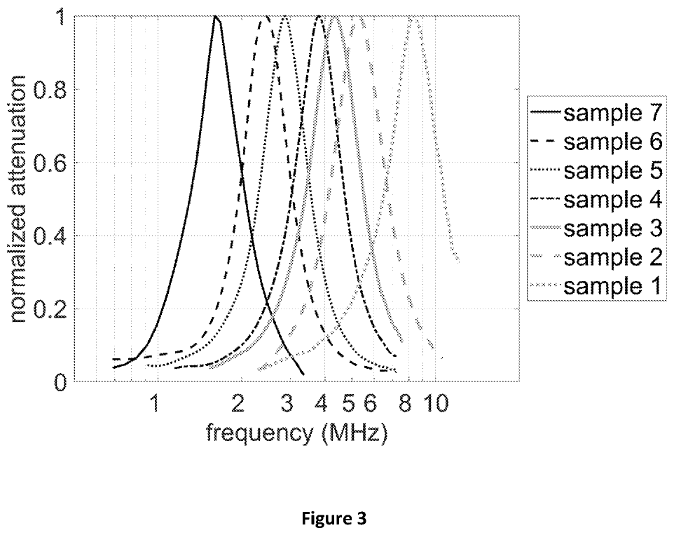

FIG. 3 illustrates the normalized attenuation for different microbubble samples.

The known unit, schematically illustrated as microbubble generation unit 1 in FIG. 1, comprises two inlets 2, 2′ through which the continuous phase fluid is fed and an inlet 3 through which the dispersed phase fluid is fed. Inlets 2, 2′ are in fluid communication with each other. Most often, a single inlet can be used, hereafter denoted as inlet 2, after which the inputted fluid can be split over the respective upper and lower channels in FIG. 1.

Due to the bends in the upper and lower channel, the continuous phase fluid impinges onto the dispersed phase fluid from two opposite sides. It thereby shapes and confines the flow of the dispersed phase fluid such that bubbles or droplets 4 of the dispersed phase fluid are formed in the continuous phase fluid inside a bubble formation channel 5. Bubbles 4 are essentially created one after the other.

Bubble formation channel 5 in FIG. 1 has a rectangular cross section, having a width in the range of 15-35 micrometer, a height in the range of 10-30 micrometer, and a length in the range of 50-1000 micrometer.

The following, non-limiting example is provided to illustrate the invention.

### Example 1

For preparing 30 ml of phospholipid solution of DPPC and DPPE-mPEG5000K with a molar ratio of 85:15 respectively, and a total mass lipid concentration of 15 mg/ml, dissolved in a liquid solution of PG and PBS with a (V/V %) volumetric ratio of 5:95, the following ingredients were weighted out:
0.189 g of DPPC
0.261 g of DPPE-mPEG5000K
1.5 g of PG
35-28.4 g of PBS.

PG and PBS were preheated to 74° C. in separate round-bottomed flask. In this case first DPPC was added and dissolved in the preheated PG, and after it was completely dissolved, DPPE-mPEG5000k was added to the preheated PG solution comprising the dissolved DPPC. After achieving complete solubilization of the lipids in the PG, the preheated PBS was added. The resulting solution was stirred at 74° C. overnight, and filtered using a 0.22 μm cellulose acetate membrane.

The final phospholipid solution was stored and cooled to room temperature, ready for use.

Using this phospholipid formulation and a flow focusing microfluidic device, seven microbubble samples were produced using different gas-to-liquid flow rate ratios. Microbubbles were collected in the collection reservoir designed for this purpose. Particle size standard analyser Coulter Counter (Beckman) was used to characterize the size of each microbubble sample, obtaining the results as summarized in Table 1.

TABLE 1

| | Mode diameter | PDI | Resonance frequency |
|---|---|---|---|
| | (μm) | (%) | (MHz) |
| sample 1 | 1.9 | 5.6 | 8.4 |
| sample 2 | 2.7 | 7.5 | 5.3 |
| sample 3 | 3.1 | 7.2 | 4.3 |
| sample 4 | 3.5 | 6.8 | 3.8 |
| sample 5 | 4.2 | 7.2 | 2.9 |
| sample 6 | 4.5 | 7.8 | 2.4 |
| sample 7 | 5.8 | 6.7 | 1.7 |

Attenuation measurements were furthermore performed to measure the resonance frequency. For mono-disperse microbubbles the resonance frequency corresponds to frequency of the peak value in the attenuation curve. The results are given in the FIGS. 1 and 2.

FIG. 1 shows the size distribution of different microbubble populations. As can be concluded from the Figure, the size distribution of the microbubbles is narrow, and no coalescence of microbubbles, resulting in polydisperse microbubbles, has taken place.

FIG. 2 shows the normalized attenuation for different microbubble samples. The resonance frequency corresponds to the peak value in the attenuation curve. The resonance frequency is linear dependent on the inverse of the microbubble diameter.

Overall it is demonstrated that the process of the invention to produce a phospholipid composition is successful and that a phospholipid composition can be prepared with a high concentration of phospholipids, that can be suitably used in a system for controlled manufacturing of microbubbles.

In the above, the invention has been disclosed using examples thereof. However, the skilled person will understand that the invention is not limited to these examples and that many more examples are possible without departing from the scope of the present invention, which is defined by the appended claims and equivalents thereof.

1. A process for preparing a hydrated phospholipids solvent mixture, by:
dissolving a first phospholipid at a temperature above the phase transition temperature of the phospholipids in an organic solvent to form a dissolved phospholipid solvent mixture;
dissolving a second phospholipid at a temperature above the phase transition temperature of the phospholipids in the dissolved phospholipid solvent mixture to form a dissolved phospholipids solvent mixture;
adding an aqueous phosphate buffer to the dissolved phospholipids solvent mixture to form a buffered phospholipids solvent mixture; and
stirring the buffered phospholipids solvent mixture to form a hydrated phospholipids solvent mixture,
wherein the phospholipids are a combination of the first phospholipid being at least one out of the group of DPPC, DSPC, DSPG, DMPC, DBPC, DPPE and the second phospholipid being at least one out of the group of DPPE-mPEG5000, DMPE-PEG-2000 and DSPE-PEG2000, wherein the concentration of the phospholipids in the hydrated phospholipids solvent mixture is in the range of from 5 up to 20 mg/ml, wherein the ratio of the first phospholipid and the second phospholipid is in the range of from 95:5 to 70:30, and wherein the hydrated phospholipids solvent mixture comprises no dipalmitoylphosphatidic acid.

2. The process of claim 1, wherein the hydrated phospholipids solvent mixture is filtered over a sterilization filter to form a sterilized hydrated phospholipids solvent mixture.

3. The process of claim 1, wherein the phospholipids are a combination of the first phospholipid being at least one out of the group of DPPC, DSPC, DPPE and the second phospholipid being at least one out of the group of DPPE-mPEG5000 and DSPE-PEG2000.

4. The process of claim 1, wherein the organic solvent is selected from the group of propylene glycol, ethylene glycol, polyethylene glycol 3000 and/or glycerol.

5. The process of claim 1, wherein the aqueous phosphate buffer is phosphate buffered saline (PBS), phosphate buffered saline with glycerine, water, saline, saline/glycerine and/or a saline/glycerine/non-aqueous solution.

6. The process of claim 1, wherein the buffered phospholipids propylene glycol mixture is stirred for at least 1 hour.

7. The process of claim 2, wherein the sterilization filter has a pore size of 0.2 micrometer.

8. The process of claim 1, wherein all process steps are performed at a temperature above the phase transition temperature of the phospholipids.

9. The process of claim 1, wherein the concentration of the phospholipids in the hydrated phospholipids solvent mixture is in the range of from 10 up to 18 mg/ml.

10. The process of claim 1, wherein the ratio of the first phospholipid and the second phospholipid is in the range of from 90:10 to 75:25.

11. The process of claim 1, wherein sequentially one or more phospholipids are dissolved in the dissolved phospholipids solvent mixture at a temperature above the phase transition temperature of the phospholipids.

12. Use of the microbubbles manufactured using a system for controlled manufacturing of microbubbles, comprising: a microbubble generation unit having a first inlet for receiving a dispersed phase fluid, a second inlet for receiving a continuous phase fluid, and a bubble formation channel in which microbubbles are generated using the received dispersed phase fluid and the received continuous phase fluid, wherein the continuous phase fluid is the phospholipid composition obtainable by the process according to claim 1 for ultrasound examination.

13. Use of the microbubbles manufactured using a system for controlled manufacturing of microbubbles, comprising: a microbubble generation unit having a first inlet for receiving a dispersed phase fluid, a second inlet for receiving a continuous phase fluid, and a bubble formation channel in which microbubbles are generated using the received dispersed phase fluid and the received continuous phase fluid, wherein the continuous phase fluid is the phospholipid composition obtainable by the process according to claim 1 for therapeutic applications.
